(19) United States
(12) Patent Application Publication
(10) Pub. No.: US 2026/0237064 A1
(43) Pub. Date: Aug. 13, 2026

(54) MOBILE DOCUMENTATION DEVICE FOR DETECTING SKIN LESIONS

(71) Applicant: CUREVISION GMBH, Muenchen (DE)

(72) Inventors: Johannes RUOPP, Muenchen (DE); Richard FOBO, Muenchen (DE)

(21) Appl. No.: 19/156,270

(22) PCT Filed: Feb. 14, 2024
(86) PCT No.: PCT/EP2024/053763

§ 371 Date: Aug. 13, 2025

(30) Foreign Application Priority Data
Feb. 14, 2023 (DE) 10 2023 103 577.0

(51) Int. Cl. G06T 7/00; G16H 15/00; G16H 30/40; G16H 40/67; G16H 50/20

(52) U.S. Cl. CPC G06T 7/0012; G16H 15/00; G16H 30/40; G16H 40/67; G16H 50/20; G06T 2207/10004; G06T 2207/30096

(57) ABSTRACT

The present invention relates to a mobile documentation apparatus for data detection of skin lesions, for example, wounds, which comprises a plurality of recording modules. The recording modules each comprise at least one camera for detection and at least one light source for illuminating the skin lesion. The recording modules and/or the mobile documentation device are not connected to each other in a circumferentially closed structure, i.e., not as a ring, ellipse, rectangle, each with a central opening, or the like. The invention further relates to a system.

### FIELD OF THE INVENTION

The present invention relates to a mobile documentation apparatus, a system, and a method for mobile detection of skin lesions.

### BACKGROUND OF THE INVENTION

When documenting healing-specific parameters of wounds or skin lesions, a two-dimensional image of the wound or skin lesion is usually taken with a conventional digital camera. A ruler held in the image is intended to enable an objective assessment of the wound size.

Wound care for a patient is often carried out by a number of different medical professionals and/or over long periods of time. For this reason, the course of treatment must be documented as accurately as possible so that all persons or professionals involved may reliably assess whether the treatment measures taken so far are/were successful or whether complications have arisen.

### SUMMARY OF THE INVENTION

An object of the present invention may be to specify a further documentation apparatus that enables reliable and accurate documentation of the healing processes of wounds and other skin lesions. Furthermore, a system and a method are to be proposed.

The object according to the present invention is achieved by a mobile documentation apparatus with features as described herein, as well as by a system with the features described herein and a method with the features described herein.

The present invention relates to a mobile documentation apparatus for the detection, data detection, evaluation, three-dimensional visual representation, and/or documentation of skin lesions, for example wounds.

The mobile documentation apparatus (hereinafter also referred to as: documentation apparatus) comprises one or more recording modules (or exactly one such module), or is such a module, which in turn each comprise either at least one, mostly optical, camera and optionally at least one light source and/or light source in the UV/IR range, or optionally at least one thermal imaging camera, a hyperspectral camera and/or an IR camera.

The light source, if present, serves to illuminate the wound or skin lesion (hereinafter referred to as wound) preferably without shadows. This allows images to be obtained regardless of the lighting conditions of the environment and/or under reproducible lighting conditions, for instance by the optional optical camera (a).

The recording modules, or their housings, of the mobile documentation apparatus are optionally connected to each other by one or more joints, e.g. in order to be able to assume a completely or essentially arcuate arrangement. In this case, the recording modules or the housings accommodating them are not arranged in a circumferentially closed structure, i.e., they are not connected in the form of a ring, an ellipse, a polygon, each with a central opening, or the like. Alternatively, the recording modules, or their housings, are not hinged to each other.

Optionally, the mobile documentation apparatus does not have a closed-perimeter structure.

In use, the mobile documentation apparatus is positioned over the affected wound of the patient, for example, set up or held by the user over the affected area.

According to the present invention, a system is further proposed which comprises one or more mobile documentation apparatuses, which are preferably according to the present invention.

In addition to at least one mobile documentation apparatus, the system comprises a data storage device which is used to store images or image data of wounds of one or more patients. The images and/or other patient data may have been taken using the same mobile documentation apparatus or with different devices, which are preferably identical in construction to each other.

A calculation unit for processing images or image data taken by the documentation apparatus or for processing other information is also encompassed by the system. The calculation unit may be, or encompass, a control device, such as the one disclosed herein.

Furthermore, the system comprises an output device, wherein a device of the system, e.g. the control device, is programmed to output information relating to the images taken.

The method according to the present invention relates to the use of a documentation apparatus according to the present invention or system for detecting data of a skin lesion or wound.

Embodiments according to the present invention may comprise some, several or all of the following features in any combination, unless the person skilled in the art recognizes a specific combination as technically impossible. Advantageous developments of the present invention are also described herein.

In all of the following statements, the use of the expression “may be” or “may have” and so on, is to be understood synonymously with “preferably is” or “preferably has”, and so on respectively, and is intended to illustrate embodiments according to the present invention.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present invention as the interpretation that a numerical word, for example, “one” (or “a/an”) may alternatively mean “exactly one”, wherever this is evidently technically possible in the view of the person skilled in the art. Both of these understandings are encompassed by the present invention and apply herein to all used numerical words.

Advantageous developments of the present invention are also described herein.

Whenever an embodiment is mentioned herein, it represents an exemplary embodiment according to the present invention.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

Whenever reference is made to “programmed” or “configured” it is also disclosed that these terms may be used interchangeably.

In some embodiments, there are also no connectors or other arrangements to bring the receiving modules or the housings that receive them into a closed arrangement along the circumference of the mobile documentation apparatus.

In several embodiments, the mobile documentation apparatus has a user interface (GUI/graphical user interface) or a display. The user interface (GUI) or display may, in certain embodiments, be embedded or incorporated in the outer circumference of the arcuate or trapezoidal housing. The user interface or display may be connected to an optional evaluation device, of which there may respectively be one or more herein, and/or the recording modules and/or sensors and/or light sources and/or other components in the inner circumference of the housing so that measurements, data, images, etc. may be displayed on the display and/or all components can preferably be controlled by the user interface.

In some embodiments, the GUI is used to operate or control the mobile documentation apparatus and/or to enter required information. By using the user interface or GUI, one, some, or all of the functionalities of the mobile documentation apparatus described below may be implemented, wherein the necessary equipment, programming, etc. are available, for instance on the mobile documentation apparatus or at another site in the system according to the present invention.

In several embodiments, the mobile documentation apparatus comprises several cameras, optionally one or more light sources, and optionally one or more distance sensors. All of these may optionally be provided in a common housing.

In several embodiments, the mobile documentation apparatus comprises at least one light source for some or all of the cameras on both sides thereof, i.e. e.g. in front of and behind them, or to the left and right of them, which counteracts shadow formation and may contribute to better recording or model quality.

In some embodiments, the central housing section, also referred to herein as the body of the housing, comprises one, two or more of the cameras mentioned herein and optionally one or more sensors, such as e.g. a distance sensor.

Cameras, distance sensors, and/or other sensors may each be aligned toward the same side of the mobile documentation apparatus, i.e., e.g., toward the underside of the mobile documentation apparatus and preferably toward the wound, respectively.

In several embodiments, a battery is provided for operating the mobile documentation apparatus. Alternatively, a rechargeable battery is provided, which comprises a charging option known from the prior art. The battery or rechargeable battery may be embedded in the housing.

In some embodiments, the mobile documentation apparatus comprises at least one handle with which the mobile documentation apparatus can be when used held over the wound. Alternatively or additionally, finger grips, finger recesses, and/or the like may be provided.

Recesses or handles for holding the mobile documentation apparatus as described herein may be provided in some embodiments and/or may be suitable for enabling the mobile documentation apparatus to be held and/or operated with only one hand.

The recesses described herein may advantageously allow this. The second, free hand may be used to operate the mobile documentation apparatus, e.g., to initiate the cameras to take pictures. In this, gesture control may be used, which may counteract or prevent blurring of the images caused by actuations e.g. on the mobile apparatus to trigger itself.

A storage device, if provided, may be part of the mobile documentation apparatus or part of the system which comprises the documentation apparatus.

The storage device and the control device may be connected wired or wirelessly, at least during use of the documentation apparatus, and may be in signal communication to each other or prepared therefor.

When a signal communication or communication connection between two components is mentioned herein, this may be understood to mean a connection that exits during use. It may also be understood that a preparation for such a (wired, wireless or otherwise implemented) signal communication exists, for example by coupling both components, for instance by pairing, etc.

Pairing is a process that takes place in connection with computer networks in order to establish an initial link between computer units for the purpose of communication. The best-known example of this is the establishing of a Bluetooth connection, by which various devices (e.g. smartphone, headphones) are connected to each other. Pairing is sometimes also referred to as bonding.

In a further embodiment, the images taken by the recording modules and the measured/collected parameters are stored; a data storage device designed and prepared for this purpose is provided in such embodiments. The data storage device may be part of the mobile documentation apparatus or of the system.

In some embodiments, the mobile documentation apparatus according to the present invention comprises at least one switching device by which the recording modules or their cameras and/or sensors are operated. The switching device may be an actuating device and may also be designated as such.

The recording modules or their cameras and/or sensors may be operated simultaneously, the control device may be programmed accordingly. Alternatively, successive actuation or activation of the individual recording modules or cameras and/or sensors, with or without a predetermined sequence, i.e., in any sequence, or in a predetermined sequence, is also encompassed by the present invention.

In several embodiments, at least one of the recording modules comprises at least one thermal imaging camera. This may be provided in additionally or alternatively to the optical camera and/or other cameras disclosed herein.

In several embodiments, at least one of the recording modules further comprises at least one sensor, in particular a temperature sensor, an IR sensor, one or more UV cameras or sensors, and/or a color sensor.

In certain embodiments, the sensor technology is designed to determine or detect further parameters that provide an indication of the development of the healing process of the wound under observation. These parameters may include qualitative or quantitative statements on the presence of histological observations such as fibrin, granulation tissue, necrotic tissue, etc.

According to the present invention, the mobile documentation apparatus may have more than one type of sensor to be able to make more accurate statements.

For example, a temperature increase measured by an optional temperature sensor, for example over different examination times at which the documentation apparatus according to the present invention was used, may be an indication of a complication of the wound (inflammation or infection), or alternatively it may also be an indication of revascularization of the tissue that is favorable for healing, if ischemia (reduced blood flow) existed previously.

Additional sensors, such as IR sensors or color sensors, may be provided to supply further information in accordance with the invention and, for example, to expand or supplement the information obtained from the temperature sensor(s) such that the clinical value of the temperature change may be determined more accurately.

The values determined by the sensors may provide information on the healing process (documented over longer periods of time), e.g., with regard to the formation of granulation tissue or epithelialization of the wound surface, or the development of complications such as infection(s), poor blood circulation, in the worst case necrosis, etc.

In some embodiments, at least one of the recording modules and/or another section of the documentation apparatus comprises at least one distance sensor, in others exactly one, and in yet others exactly or at least two distance sensors.

In several embodiments, the distance sensor may determine the correct positioning of the mobile documentation apparatus, for example so that it is always placed at the same site on the patient during successive measurements. The control device may accordingly be designed and/or programmed for this purpose.

In further embodiments, the distance sensor(s) may serve for this purpose or exclusively for this purpose, for example, to produce lifelike 3D images of the wound or to contribute to this, for example, by determining the depth of the wound or by enabling correct reconstruction of the wound, e.g., in a 3D data model, since their sensor values can allow conclusions to be drawn about the position of the cameras in the space or relative to each other at the moment of recording. An algorithm used e.g. in the optional evaluation device may make use of the distance values, for instance when creating 3D models, views, evaluations, or other activities. The same may apply to the optional inclination sensors mentioned below.

In some embodiments, at least one of the recording modules and/or another section of the mobile documentation apparatus comprises at least one inclination sensor. Inclination sensors may be provided to give the user indications when holding the mobile documentation apparatus as to how the latter is being held at that moment. A comparable principle is known from spirit levels. One or two inclination sensors indicate whether the spirit level is aligned “water level” in one or two spatial directions. A mobile documentation apparatus with inclination sensors may prompt the user, through visual or other feedback, to hold the documentation apparatus straight or at a constant angle at least during each examination of the wound at different times. The mobile documentation apparatus, which is kept identical or at least comparable in at least one or more spatial directions, allows images to be taken at different times, which enables the reconstruction or creation of 3D models with comparatively less computing effort. This applies all the more if the height of the mobile documentation apparatus above the wound at the moment when the respective image was taken is known based on data from the distance sensor, or if the mobile documentation apparatus is held in accordance with the specifications of the distance sensor or the information determined by it.

In further embodiments, recording modules may be combined, i.e., have different camera and/or sensor types.

In several embodiments, the mobile documentation apparatus or its recording modules have a control device or are in signal communication therewith during use.

In further embodiments, the control device is configured for automatic or manual control or closed-loop control of the function of the camera(s) or of the sensor(s).

In some embodiments, the control device is configured to automatically or manually calibrate cameras and/or sensors of the mobile documentation apparatus and/or to adjust, influence, and/or change setting parameters of the cameras and/or sensors, such as angular positions, for example relative to the wound or relative to each other.

In some embodiments, the control device is configured to adjust for example an angle setting of the camera(s) and/or of the sensor to the corresponding position on a patient's body or to the specific area and depth of the wound with the aim of ensuring the best possible camera images or measurement values from the sensor(s).

In further embodiments, the control device may adjust the light source(s) so that the wound is illuminated substantially without shadows.

In some embodiments of the mobile documentation apparatus, the intensity and/or the orientation and/or the dimensions (size and shape parameters) of the light cone of the light source may be adjustable manually or automatically.

In several embodiments, the mobile documentation apparatus comprises an evaluation device or is connected to an evaluation device. In some embodiments, the evaluation device lies elsewhere in the system, but not on the mobile documentation apparatus.

The evaluation device is preferably configured or programmed to determine or collect data, convert the data determined in this way into images, and/or compare these images with other images that were calculated in particular based on previous data.

In some embodiments, the evaluation device is configured to create three-dimensional models of wounds based on the data determined by the mobile documentation apparatus.

The model created by the evaluation device may, for example, reproduce physical parameters of the wound, such as e.g. area and depth, and/or physiopathological characteristics of the wound, such as e.g. temperature, discoloration, tissue vitality signs, etc., which may be represented for example on the model as numbers or color code.

3D models (or their mathematical description, their image, etc.) taken or generated on different days are stored in some embodiments. In several embodiments, they are compared with each other upon initiation of a user or automatically, for instance so that a chronological documentation of the course of the wound is created.

“Determining” or identifying, in particular data and/or values, may encompass examining existence or non-existence, recording, measuring, collecting, evaluating, processing, comparing, estimating, assessing, assuming, calculating, obtaining, achieving, and/or recognizing.

In some embodiments, the evaluation device may make prognostic statements regarding the course of the healing process or may trigger an alarm message if signs of a complication in wound healing have occurred. This can be done, for example, by comparison with stored reference data, reference histories, reference images, by using reference databases, using artificial intelligence, etc.

In several embodiments, the evaluation device may be designed as a user interface, be connected to such an interface preferably wireless or wired-or have such an interface by which the cameras, sensors, light sources, and/or control devices of the recording modules may be controlled. In some embodiments, it may also comprise the switching device or be in signal communication therewith.

In several embodiments, the evaluation device may be controlled by voice and/or gestures and may be designed or programmed accordingly.

In some embodiments of the mobile documentation apparatus, the evaluation device is integrated into at least one of the recording modules or into the housing of the mobile documentation apparatus.

In several embodiments, at least the evaluation device comprises a display for data reproduction and/or for controlling the mobile documentation apparatus and/or as a user interface for entering control signals for controlling the mobile documentation apparatus.

In some embodiments, the display is a touchscreen.

In some embodiments, the at least one evaluation device is physically separate from the mobile documentation apparatus, is in wireless signal communication therewith, or is prepared therefor. Optionally, the evaluation device is configured to remotely control the documentation apparatus.

In several embodiments, the evaluation device may exchange data with remote computer systems and be prepared or programmed accordingly and/or be in signal communication or be prepared therefor in order to enable for example remote diagnostics.

In some embodiments, the evaluation device is part of the set or system according to the present invention. It may be provided on the documentation apparatus or externally therefrom.

In several embodiments, the mobile documentation apparatus may be sterilized or disinfected.

In some embodiments, the evaluation device is programmed to prompt both the first camera to take a 3D image and the second camera to take a 2D image.

In several embodiments, the 3D image is, or results in, a point cloud.

In some embodiments, the evaluation device is programmed to prompt both images, i.e., the 3D and 2D images, to be taken after the user activates only one triggering device, for example, after activating only one triggering button, or after the user performs only one gesture

In several embodiments, the evaluation device is programmed to prompt both images, i.e., the 3D and 2D images, to be taken simultaneously or at least with a time overlap.

By initiating the 2D and 3D cameras simultaneously, it may be ensured that the wound recognized in the 2D image and the 3D model from the 3D image match. This allows the system to achieve a high degree of accuracy.

In some embodiments, the evaluation device is programmed to determine a mask from the 2D image or its underlying information, in particular image information, which mask corresponds, or is assigned, to a section of the wound, preferably to the edge of the wound, in the 2D image, wherein an inner area of the mask is itself identical to this surface.

In several embodiments, the mask is a binary mask (black/white; 0/1; etc.). In this, the assignment may be made by binary segmentation, i. e. e.g., wound=white, non-wound=black.

In several embodiments, the mask is a contour or boundary, e.g., comprising a width, depth, or thickness of one pixel.

In some embodiments, the mask comprises a circumferentially closed structure that may define an inner area of the mask.

The mask may be obtained by morphological erosion and subtraction from the initial image or the original.

In some embodiments, the evaluation device is programmed to define an area of the 3D image which, after or by projection of the mask onto the 3D image, is assigned to the edge or its course or the to the inner area of the mask, or lies within the contour or the boundary of the mask or is assigned to this, if applicable, circumferentially closed area.

In several embodiments, the evaluation device is programmed to extract the previously defined area of the 3D image, or an area defined by the contour or boundary, from the 3D image.

In some embodiments, the evaluation device is programmed to evaluate the extracted area.

In several embodiments, the evaluation is, or encompasses, defining a plane in the 3D image which comprises the defined edge, or at least three pixels which lie on or at the edge.

In some embodiments, methods or algorithms may be used for this purpose, by which the shape or orientation of points may be determined from a point cloud and, based on this, a plane may be calculated in which most of these points lie. Such methods/algorithms are known to the skilled person. Reference is also made to method of least errors or methods of least squares.

In some embodiments, this plane may be a wound entry plane within the 3D image.

In several embodiments, the evaluation is, or encompasses, determining the distances from a plurality of pixels of the 3D image to the plane surrounding the edge.

In some embodiments, these distances may be or become color-coded.

In several embodiments, this may be used to generate a depth map.

Methods for calculating distances between individual points, herein for example the wound base, and a surface, herein for example the wound entry surface, are known to skilled person.

In some embodiments, the evaluation is, or encompasses, determining the area of the wound base underlying the surface using the determined distances.

In several embodiments, the evaluation is, or encompasses, measuring the extracted area.

In some embodiments of the mobile documentation apparatus or the system according to the preset invention, the output information is, or includes, information about at least one dimension of the wound, such as e.g. depth, width, length, area, number of pixels, pixel content, etc.

In several embodiments of the mobile documentation apparatus or system, the information is, or includes, information about at least one, in particular histological, finding of the wound, such as the presence and/or proportion of tissue types or cell types, their quality or quantity, or the like.

In some embodiments, the calculation unit of the system has or uses algorithms for image processing and/or image comparison, in particular those based on artificial intelligence.

In several embodiments, the calculation unit is, or comprises, the evaluation device mentioned herein, and the reverse case, according to which the at least one evaluation device is the calculation unit or comprises it, is also covered by the present invention.

In some embodiments, the control device is the evaluation device mentioned herein, and/or vice versa.

In several embodiments, the mobile documentation apparatus or system encompasses a function for authenticating the user.

In some embodiments, the mobile documentation apparatus or system encompasses a function for identifying or assigning the patient.

In several embodiments, the mobile documentation apparatus or system encompasses the possibility of selecting the wound to be documented, in particular whether and, if so, optionally when the wound has already been documented at an earlier point in time. This is done in particular in order to subsequently be able to relate and compare the images or models of the same wound obtained at different times with each other. In this way, the wound healing process can be documented in detail.

In some embodiments, the mobile documentation apparatus or system encompasses a possibility for entering wound details (score, genesis, etc.) and/or wound treatment measures. Such entries may refer to, for example, a classification of the wound, which may reduce the computing capacity required in the following. In this way, measures taken on the wound (e.g., dressings, medication, etc.) could be documented in detail and their effectiveness assessed. An effective measure can thus be stored, preferably in a retrievable form, for example in the system's data storage, so that it can then be issued, for example, as a treatment recommendation, in particular for similar wounds in other patients.

The individual components of the system may be prepared or set up in any combination to be in signal connection with each other.

In several embodiments, a camera preview may be displayed on the GUI or elsewhere, which may be advantageously used to guide the user in positioning the mobile documentation apparatus over the wound.

In some embodiments, a triggering button for camera capture is implemented on the GUI.

In several embodiments, the image taken (e.g., by the main camera, or a 3D model, or the like) of the wound may be displayed on the GUI or elsewhere on the documentation apparatus or system for inspection by the user. The user may then check the image for errors or blurring, for example. In certain embodiments, said check may be performed or supported by the mobile documentation apparatus or system. If, for example, the image quality of the image is insufficient, which may be determined objectively or subjectively, e.g., by comparison or key figures, it may be provided at this point to repeat it. The user may, supported by the mobile documentation apparatus, receive appropriate instructions for this.

In some embodiments, the input of additional documentation parameters via the GUI may be provided as an option. The documentation parameters are, or encompass, for example, information on wound type, wound classification, undermination, wound odor, wound infection, wound pain, wound location, exudate, wound edge, wound environment, comments on medical history, treatment and diagnosis, and/or the like.

In several embodiments, the GUI may also be used to access the hardware elements in order to control or operate them. In particular, this can enable one, several, or all of the following controls:
Control of the individual camera modules (image capture and communication with master circuit board), for example, in order to trigger the existing cameras synchronously (in particular simultaneously, within a predetermined period of time or at predetermined intervals) and then store the captured images in the data storage
Control of the main camera and the image parameters that are necessary for reproducible image capture (e.g., white balance, exposure, etc.)
Scanning the distance sensors to determine and store a distance value
Communication with the backend, i.e., for example, with the system server, the data storage for captured images, a cloud, etc.

In some embodiments, the software of the mobile documentation apparatus or the system for evaluating the images taken by the documentation apparatus encompasses algorithms and analysis techniques “advanced analytics” based on artificial intelligence (AI), in particular radiomics, neural networks, mathematical functions, and/or the like. These may encompass, among other things, evaluations of images taken using statistical and/or learning methods.

The term “advanced analytics” may include the semi-automatic or automatic analysis of data. This involves the use of sophisticated prior art methods and tools, e.g., regression, clustering, cohort analysis, recommendation algorithms, optimization, e.g., Bayesian optimization, gradient boosting (e.g., the XGBoost library), (decision) tree algorithms, preferably directed and/or acyclic, influence diagrams, graphical probability models, or the like.

In some embodiments, the term “advanced analytics” encompasses in particular methods that enable models to be derived based on empirically determined data for making assessments and predictions.

Alternatively or in addition, for example various data mining methods, can fall under the term “advanced analytics.” The term “data mining” encompasses the semi-automatic or automatic evaluation of large amounts of data (which, for example, were obtained from patient groups) to determine occurring regularities, patterns, and hidden connections, from which conclusions can be drawn about expected developments, herein wound healing. The term “data mining” can in turn be encompassed by the broader process of knowledge discovery in databases (KDD).

In several embodiments, “advanced analytics” include algorithms based on neural networks and/or machine learning, in particular “deep learning” and/or “reinforcement learning.”

Neural network architectures may encompass convolutional neural networks (CNN), networks with long-short-term memory networks, recurrent neural networks, or other types of neural networks that are commonly used for optimization or classification problems and are known in the prior art.

In several embodiments, the calculation unit is configured to assign a diagnostic and/or prognostic value to the input data in an optional additional evaluation step. The input data may encompass, for example, the results of an examination or diagnosis of the respective wound by the user or automatically, individual physiological patient data, systemic diseases such as diabetes mellitus, and/or drug data relating to the previous treatment of the respective wound (medications, dressings, etc.). Alternatively, the calculation unit is configured so that it assigns a diagnostic and/or prognostic value to the input data in its entirety, likewise in the evaluation step.

The diagnostic and/or prognostic value can be the probability of a specific outcome, for example a timing for complete healing of a wound, a timing for a specific treatment procedure, a timing at which deterioration of the patient's condition is to be expected, or the like. The value may be, or encompass, a risk assessment, an objective medical assessment, etc. Alternatively or additionally, this value may also indicate a deterioration, an emergency, or the like.

For example, an image of a wound taken using the mobile documentation apparatus may be designated with three out of five possible points. Five points means that the wound is in perfect condition and zero points means that the wound, and in particular the condition of the vessels in the wound, is in the worst possible condition.

Similarly, clinical staff can assess the wound with the naked eye and also designate a number of points (e.g., up to a maximum of five possible points). In some embodiments, it may be possible to enter this score via the mobile documentation apparatus, for example via its display.

Similarly, a corresponding value may be assigned to any other data obtained from the image of the wound or entered by the user.

Alternatively or in addition, a value may generally be assigned to an input. This value may be encompassed in an algorithm that takes several values into account (one, for example, from the assessment of medical staff, another, for example, from the analysis of the taken image, etc.) or it may be obtained in another way, under consideration of all available data.

In several embodiments, the software of the mobile documentation apparatus or system or the methods used encompass binary segmentation, multi-class segmentation, and/or depth measurement. This allows, for example, different types of tissue in the wound area to be distinguished or progress in wound healing to be detected.

In some embodiments, the calculation unit is part of the mobile documentation apparatus, in others it is part of the system but not part of the mobile documentation apparatus. The calculation or evaluation of the captured images and the information obtained from them may thus be performed in several embodiments on the mobile documentation apparatus, in others it is performed at another site in the system.

Optional preprocessing of the captured images or image data on the calculation unit may result in the camera section actually encompassing the image of a wound, preferably in its entirety, or not.

An optional processing performed on the calculation unit, for example evaluating the captured images or image data, for instance using AI, may be used to classify the wound based on scores, previous images, histological observations, such as the qualitative and, if applicable, quantitative presence of predetermined tissue types such as fibrin, granulation tissue, necrotic tissue, etc., or may encompass such classification. In some embodiments, this classification may provide, for example, information about the expected further course of wound healing and/or help to assess the need for treatment measures, for example the need for medication, wound care, surgery, and/or the like.

An optional postprocessing, performed on the calculation unit, uses the information obtained from the captured image and/or the results of the data processing carried out up to that point, in particular by mathematical algorithms, to generate results that may be used by the user (e.g., length of the wound, width, wound healing process, etc.) and/or treatment suggestions (e.g., medication, care, surgery, etc.). These results, like all other results of the information and findings obtained by the calculating unit, may be communicated to the user, e.g., in words or graphics, in particular graphically, or in another manner. The control device may be programmed to make this available by the output device, which may be a display, a printer, and/or the like.

A programming for issuing therapy recommendations via the mobile documentation apparatus or system is also provided in several embodiments.

In some embodiments, a recording module is, in the simplest case, a camera. In other embodiments, a recording module is a combination of mostly electronic components such as a camera, sensor, etc., in particular as disclosed herein. The assembly may be provided on a carrier material such as a circuit board, a template, an insert, etc. Therefore, in such cases, one may denote the term recording module to the carrier material equipped with the components. The carrier material extends in a main extension plane. If two adjacent recording modules or their main planes of extension are at an angle (unequal to 0°) to each other, this may refer, for example, to the center lines of their camera angles, which are then not parallel to each other, or to the main planes of extension, which are then not parallel to each other.

In several embodiments, the mobile documentation apparatus does not have a marking device for marking the skin. A marking device may be a device for leaving a mark on the skin even after images have been taken. Non-directional or temporary illumination of the wound or the surrounding skin does not count as marking. The application of stamps, colors, grids, etc., on the other hand, does. Devices for applying markings, e.g., by spray, printing, or the like, count as marking devices. In some embodiments, a control device of the mobile documentation apparatus is not programmed to control such a marking device when using the mobile documentation apparatus to take pictures or recordings.

In some embodiments, the center line of the camera angle refers to an optical axis.

In several embodiments, the housing of the mobile documentation apparatus is two-part. In some embodiments, it comprises one, or exactly one, continuous upper shell and one, or exactly one, continuous lower shell. This has proven to be advantageous for sealing the mobile documentation apparatus against the entry of liquids, e.g., during sterilization. The requirements for labyrinth seals, etc., may be less.

In some embodiments, the mobile documentation apparatus is not an oral scanner.

In several embodiments, the mobile documentation apparatus does not have color light sources and/or light sources of different light qualities.

In several embodiments, sequences of images captured by the same camera are not used for evaluation, for creating a 3D model, etc.

Some or all embodiments according to the present invention may have one, several or all of the advantages mentioned supra and/or below.

Conventional documentation systems based on two-dimensional images do not allow for reliable documentation of wound progression or wound healing due to unavoidable measurement inaccuracies, especially in the case of large lesions. These images are not suitable as the sole source of assessment. Depending on the lighting, angle of the image, and position of the ruler, the informative value of such images may vary.

One advantage of the present invention may be that, due to its compact, mobile design, it is quickly available at a place of use. In some embodiments, it is approximately the size of an iPad mini or a tablet. Due to its low weight, for example less than 1.0 kg in some embodiments, it can be used directly during the examination of a patient on site. This saves the patient a trip to an examination device. This facilitates outpatient examinations.

Another advantage of the present invention may be that many crucial wound parameters may be detected simultaneously. In addition, the detected parameters may be used to create a complete 3D model of the wound, which in certain embodiments may be used to document not only document the size or extent (area/depth) of the wound, but also provide data on vitality, blood flow, inflammation, wound healing, and/or the like. This may help the treating physician to issue the correct treatment of the wound.

The present invention allows a patient's wound to be assessed or examined within a very short time. This helps to save time and costs and does not place any unnecessary strain on the patient.

Another advantage of the invention may be its user-friendliness. In certain embodiments, intuitive LEDs, for example by glowing green, indicate the correct positioning, and the triggering process itself takes approximately 0.1 seconds in certain embodiments. After approximately 30 seconds, the results can be displayed both on the device's built-in display and on the practitioner's computer, in some cases even earlier. Therapists who are under considerable time pressure may advantageously use the time saved, e.g. for analysis and therapy planning.

The ability to disinfect the present invention allows it to be used in clinical practice and may represent a further advantage.

Furthermore, the reliable, detailed detection of wound parameters even when the patient is moving may also be advantageous. This may be particularly advantageous in the treatment of chronic wounds. Chronic wounds often occur as a complication of other diseases that make it impossible for the patient to remain still for a longer imaging time, for example due to tremors. With the present invention, all parameters are captured simultaneously within 0.1 seconds, and the quality of all images is always equally good, even if the patient trembles during this time or the image is reproduced from a different camera angle.

Since, by using the integrated software, the most important characteristics of the wound (such as diameter, depth, redness, blood circulation status, vitality, temperature, inflammation, exudate, and/or the presence of specific tissue types, in particular at the wound edge, such as e.g. swelling, potential necrosis, etc.) may be automatically detected in a very short time, the present invention may also be easily used for wound assessment over time. Since medical standards require the documentation of chronic wounds at regular intervals, the present invention may also support the treating physician in this regard. In particular, the present invention may be helpful in the time-efficient and objective assessment of therapeutic success.

In addition, the invention can offer further additional benefits, such as providing prognoses based on the chronological course of the healing process. In addition or alternatively, it can send warnings, for example, if there are indications of upcoming or emerging complications, such as the development of an infection or reduced tissue perfusion. Such indications can in turn be used as a decision-making aid in treatment, for example to determine the point in time when a doctor should be involved in the treatment.

### BRIEF DESCRIPTION OF THE DRAWINGS

The present invention is described below purely by way of example with reference to the accompanying drawings. In the figures, identical reference numerals designate identical or similar components. The following applies:

FIG. 1 shows a mobile documentation apparatus in an exemplary embodiment;

FIG. 2 shows the embodiment of FIG. 1 in a perspective view obliquely from above;

FIG. 3a shows the mobile documentation apparatus of FIG. 1 tilted upward by 90° from the side;

FIG. 3b shows the mobile documentation apparatus of FIG. 1 tilted 90° to the right from the side;

FIG. 3c shows the mobile documentation apparatus of FIG. 1 tilted 90° to the left from the side;

FIG. 4 shows the mobile documentation apparatus of the preceding figures with a view of the underside;

FIG. 5 shows an exemplary mask with an inner area; and

FIG. 6 shows, schematically simplified, a system according to the present invention in one embodiment.

### DETAILED DESCRIPTION

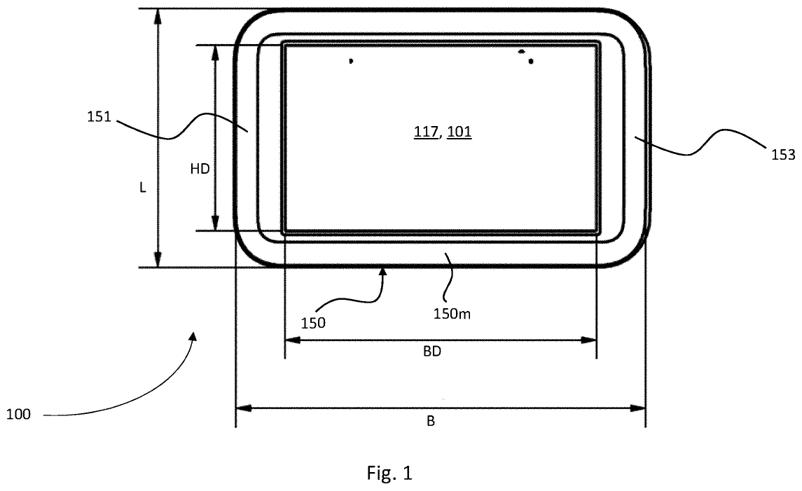

FIG. 1 shows a mobile documentation apparatus 100 according to the present invention in a first embodiment.

In the example of FIG. 1, the housing 150 can be seen, in which at least one recording module 101 may be received. The housing may, for example, have the dimensions 211.89 mm (width B) by 133.57 mm (length L), but other dimensions are also possible.

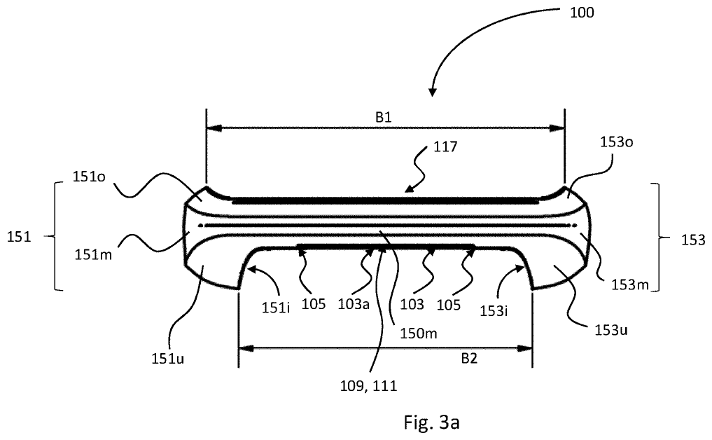

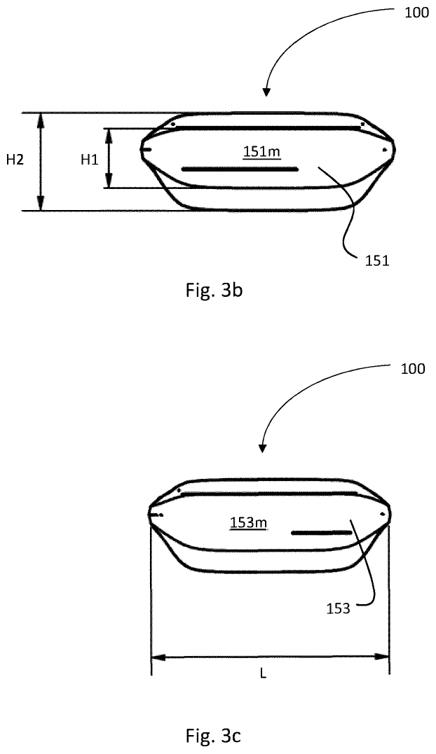

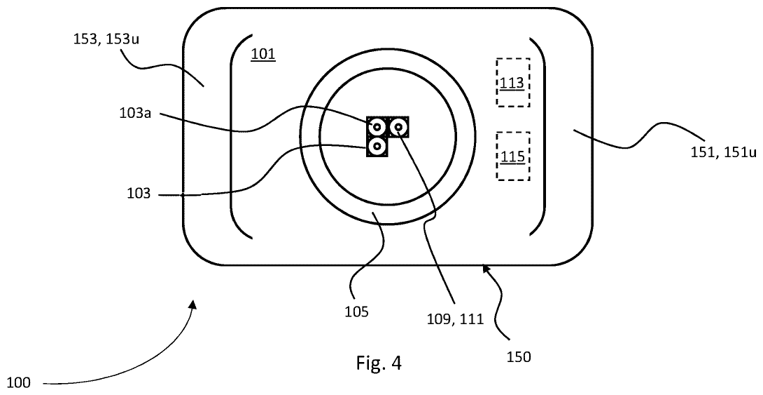

The recording module 101 may comprise at least one first camera 103 (not shown in FIG. 1, see FIG. 3a and FIG. 4), which may be a 3-dimensional camera, and at least one display 117. The display 117 may be used for data reproduction and/or control and/or as a user interface for entering control signals for controlling the mobile documentation apparatus 100.

The display 117 of the mobile documentation apparatus 100 may, for example, encompass, or be, a 5-inch, 6-inch, 7-inch, or 8-inch screen, or larger, or a size with any value between any two of the above inch values. In this, for example, the width BD of the display 117 may be 160.9 mm and the height HD of the display 117 may be 96.00 mm, but other dimensions are also encompassed.

In some embodiments, the recording module 101 may comprise the evaluation device 115 (not shown in FIG. 1).

The housing 150 of the mobile documentation apparatus 100 further comprises a left holding section 151 and a right holding section 153, which are provided and suitable for holding the mobile documentation apparatus 100 by the user. However, both the left holding section 151 and the right holding section 153 are optional.

The left holding section 151 and the right holding section 153 are connected in the example of FIG. 1 by a central housing section 150m to the housing 150 or to each other, respectively.

In the embodiment shown, the length L of the left holding section 151 and/or the right holding section 153 is optionally no longer than the width B of the mobile documentation apparatus 100. The relevant holding section 151, 153 thus advantageously does not protrude beyond the width B of the mobile documentation apparatus 100.

The mobile documentation apparatus 100 may comprise an energy supply module, which is a battery or rechargeable battery or the like, or may additionally or alternatively have the necessary connections to be connected to a voltage source e.g. the mains voltage.

If rechargeable batteries are used, they may be charged using prior art systems. Such charging stations may be provided, e.g., as part of a set, which is also covered by the present invention and already encompasses a mobile documentation apparatus 100 according to the present invention. The set may be part of the system according to the present invention.

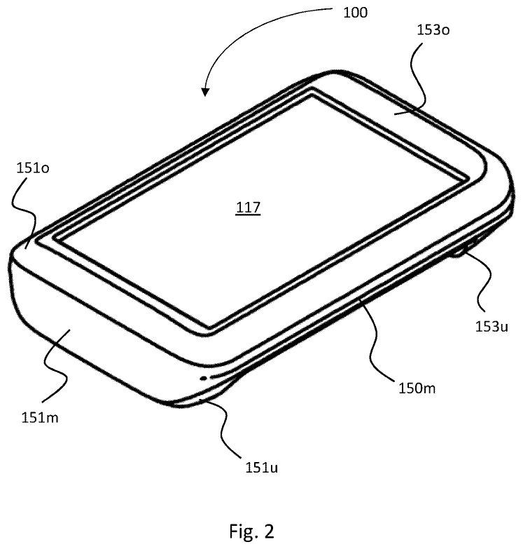

FIG. 2 shows the embodiment of the mobile documentation apparatus 100 of FIG. 1 in a perspective view obliquely from above

Reference is made to the explanations with regard to FIG. 1. The exact design of the left holding section 151 and/or the right holding section 153 is described in more detail in FIG. 3a und 3b.

FIG. 3a shows the mobile documentation apparatus 100 of FIG. 1 tilted upward by 90° from the side. In the side view of the example in FIG. 3a, due to the projection of the left holding section 151 and/or the right holding section 153, it optionally comprises both an upward and downward orientation and, in simplified terms, the shape of a large tubular bone, a double T, or an H. This design will be described in more detail below.

In some embodiments, the mobile documentation apparatus 100 comprises a second camera 103a, which may be a two-dimensional (2D) camera. The first and second cameras may be arranged anywhere on the housing, in particular on its underside facing away from the user and/or on the left holding section 151 and/or on the right holding section 153, in particular on their inner sides 151i, 153i.

In several embodiments, the mobile documentation apparatus 100 or its recording module 101 comprises at least one light source 105 for illuminating the skin lesion of interest (not shown in FIG. 3a). What is described herein for the position(s) of the first and/or second cameras 103, 103a may also apply to the position(s) of the optional light source(s) 105.

The intensity and/or orientation, or the dimensions of the light cone of the at least one light source 105 may be manually or automatically adjustable. A switch or a push button switch of any design may be provided, e.g., operable by finger or thumb, e.g., on the left holding section 151 and/or right holding section 153.

In some embodiments, the mobile documentation apparatus 100 or its recording module 101 comprises at least one thermal imaging camera (not shown in FIG. 3a). The method described herein for determining the position(s) of the first and second cameras 103, 103a and/or the light source(s) 105 described herein may also apply to the position(s) of thermal imaging camera(s).

In several embodiments, the recording module 101 of the mobile documentation apparatus 100 comprises at least one sensor 111, in particular a temperature sensor, an IR sensor, a UV camera, and/or a color sensor (not shown in FIG. 3a). What is stated here regarding the position(s) of the first and second camera(s) 103, 103a, the light source(s) 105 and/or the thermal imaging camera(s) described herein may also apply to the position(s) of the sensor(s) 111.

In some embodiments, the mobile documentation apparatus 100 further comprises at least one distance sensor 109. What is described here for the position(s) of the first and second camera(s) 103, 103a, the light source(s) 105, the thermal imaging camera(s) and/or the sensor(s) 111 may also apply to the position(s) of the distance sensor(s) 109.

Light sources, as used herein, may be sources of a visible light spectrum, alternatively or additionally sources of radiation in the UV/IR range.

It can be seen from the example in FIG. 3a that the left holding section 151 and/or the right holding section 153 optionally, here by an optional upper projection 151o, 153o project towards the user, for example via a surface, surface area, upper side or plane facing the user during use and enclosing the display 117. This may be, for example, the glass surface of the display 117, its touchscreen surface, etc. Alternatively or additionally, the left holding section 151 and/or the right holding section 153 protrudes by an optional upper projection 151o, 153o above the middle housing section 150m, which connects the left holding section 151 with the right holding section 153 or with further sections of the housing 150. This projection of the left holding section 151 and/or the right holding section 153 by an upper projection 151o, 153o may be in particular 1.5 cm or more. It serves in particular to form a handle, or part thereof, for the user's hand.

It can also be seen from the example in FIG. 3a that the left holding section 151 and/or the right holding section 153 optionally protrudes away from the user by means of an optional lower projection 151u, 153u, for example via a surface, lower surface, underside or plane facing, during use, away from the user and closing off the housing 150 or the receiving module 101 at the bottom. This may be, for example, a plane in which cameras, sensors, or light sources lie, etc. Alternatively or additionally, the left holding section 151 and/or the right holding section 153 protrudes above the middle housing section 150m by a lower projection 151u, 153u 150m, which connects the left holding section 151 to the right holding section 153 or to further sections of the housing 150. This projection of the left holding section 151 and/or the right holding section 153 by a lower projection 151u, 153u may be in particular 1.5 cm or more. It serves in particular to form a handle, or part thereof, for the user's hand.

The left holding section 151 and/or the right holding section 153 may comprise a rechargeable battery or a battery as a power source for the mobile documentation apparatus 100. arranging a rechargeable battery or a battery in one or both of the optional holding sections 151, 153 may help to save installation space or to provide an additional energy source for longer mobile use.

Furthermore, the left holding section 151 and/or the right holding section 153, in particular at its upper optional projection 151o, 153o and/or at its lower projection 151u, 153u, may comprise at least one switching device (not shown) for the user to control the mobile documentation apparatus 100, e.g. for initiating, starting or stopping a capturing by at least one of the cameras 103, 103a, for varying an illumination intensity, etc.

Such a switching device for the user may be, for example, a soft key, a push button, a slide switch, a toggle switch, or the like.

Such a switching device for the user may be arranged, or provided, on an upper side, lower side, and/or inner side 151i, 153i of the left holding section 151 and/or the right holding section 153, in particular on its upper optional projection 151o, 153o and/or on its lower projection 151u, 153u.

It can further be seen from the example in FIG. 3a that the upper projection 151o of the left holding section and the upper projection 153o of the right holding section are designed differently from the respective lower projections 151u, 153u, which results in different distances B1, B2 between the upper projections 151o, 153o and the lower projections 151u and 153u. The lower projections 151u, 153u may be more prominent or occupy more volume than the corresponding upper projections 151o, 153o, which serves to form a handle shape with which the mobile documentation apparatus 100 can be held well and in particular well and securely with one hand.

FIG. 3b and FIG. 3c show the mobile documentation apparatus 100 of FIG. 1 tilted 90° to the right and left, respectively, from the side.

It can be seen here that between the upper projection 151o, 151u and/or on the lower projection 151u, 153u of the left holding section 151 and/or the right holding section 153, there is a middle section 151m, 153m, which connects the mobile documentation apparatus 100 to its end associated with the left holding section 151 and/or the right holding section 153, i.e. forms the respective left or right edge. H1 denotes the height of the middle section 151m and H2 denotes the height of the mobile documentation apparatus 100. H1 can exemplarily be, e.g. in the present example, 31.28 mm, H2 51.38 mm, generally speaking between mm and 40 mm for H1 and/or between 30 mm and 65 mm for H2.

FIG. 4 shows the mobile documentation apparatus 100 of the preceding figures with a view of its underside in an exemplary embodiment.

In the example of FIG. 4, the first camera 103, preferably a 3D camera, and the second camera 103 a, preferably a 2D camera, are arranged on the underside of the mobile documentation apparatus 100. An optional third camera 111, which may be, for example, an infrared, hyperspectral, and/or thermal imaging camera, is also provided on the underside. In some embodiments, this camera may also be designed as a sensor or be replaced by one.

The cameras may be initiated by the evaluation device 115 optionally by the user pressing just one triggering device, such as a single trigger button, or by the user performing just one gesture, to take a picture. In some embodiments, these pictures can thus be taken simultaneously or at least overlap in time.

The number, functionality, and position of the cameras, 103, 103a, and 111 are purely exemplary and should not be understood as limiting. Any other arrangement of one or more suitable cameras is also encompassed by the present invention.

A circular light source 105 is arranged around the cameras in order to illuminate the area to be captured as completely as possible. The shape and position of the light source 105 are purely exemplary and should not be understood as limiting. Any other arrangement of one or more suitable light sources is also encompassed by the present invention.

The components 103, 103a, 105, 111 may be provided in any combination, for example as a camera with a light source, sensors with different or identical modes of operation, etc., in any order and/or arrangement, for example camera-sensor-camera or camera-sensor-sensor, etc., and/or in any number, as far as technically possible, in or on the mobile documentation apparatus 100, preferably on its underside. This is also encompassed by the present invention.

In addition to cameras 103, 103a, 111, sensors of various types can also be arranged in the mobile documentation apparatus 100, for example distance, IR, color, and/or temperature sensors as already described herein. In some embodiments, the sensors may be arranged next to, or around, the cameras 103, 103a, 111 in the mobile documentation apparatus 100.

In the example of FIG. 4, the light source 105 is represented separately from the cameras 103, 103a, 111. In other embodiments, the light source 105 may be integrated into at least one of the cameras 103, 103a, 111 and/or arranged respectively in a ring-shaped manner around the lens of the respective camera 103, 103a, 111.

As can be seen from the example in FIG. 4, the mobile documentation apparatus 100 in any embodiment may be of a size that allows it to be easily operated with two hands.

A control device 113 and/or an evaluation device 115 are purely exemplarily outlined within the housing 150 in the example in FIG. 4. The size and position of these devices are purely exemplarily and should not be understood as restrictive. In some embodiments, they may also be present outside the housing and be in signal communication with the mobile documentation apparatus 100.

The evaluation device 115 may be configured and/or programmed to collect data and/or to process and/or compare images. In addition, it may create three-dimensional models of wounds based on the data captured or measured by the mobile documentation apparatus or by its components.

The evaluation device 115 may create a chronological documentation that reflects the course of the wound healing process. It may also issue prognostic statements about this course and/or warnings if for example indications of a complication in the healing process are detected, for example indications of an infection, a wound healing disorder, tissue hypoxia, etc.

In further embodiments, the evaluation device 1 may preferably be additionally configured to control cameras/sensors 103, 103a, 109, 111, light sources 105 and/or, in some embodiments existing control devices 113 of the mobile documentation apparatus 100.

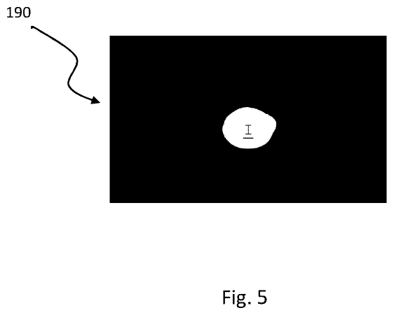

FIG. 5 shows an example mask 190 with an inner area I. The pixels of the inner area I have the pixel value 0 or white, all others have the pixel value 1 or black. The edge of the mask 190 may be the outermost row of white pixels or the innermost row of black pixels. The shape of the inner area I corresponds to the area identified as the wound area in the 2D image.

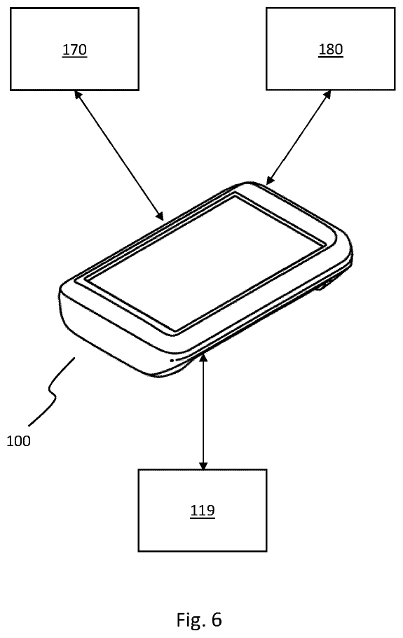

FIG. 6 shows schematically simplified a system according to the present invention in an embodiment with a mobile documentation apparatus 100, a data storage device 170 for storing images or image data of wounds of one or more patients, a calculation unit 180 for processing images or image data captured by the mobile documentation apparatus 100, and an output device 119.

### LIST OF REFERENCE NUMERALS

100 mobile documentation apparatus
101 recording module with integrated evaluation device, main module
103 first camera
103a second camera
105 light source
109 sensor (distance)
111 third camera or sensor (temperature, IR, or color sensor)
113 control device
115 evaluation device
117 display, user interface (GUI), touchscreen
119 output device
150 housing
150m middle housing section
151 left holding section
151o upper projection
151m middle housing section of the left holding section
151u lower projection
151i inner side of the lower left projection
153 right holding section
153o upper projection
153m middle housing section of the right holding section
153u lower projection
153i inner side of the lower projection
170 data storage
180 calculation unit
190 mask
B width of the housing
BD width of the display
B1 distance between the upper projections
B2 distance between the lower projections
H1 height of the middle section
H2 height of the mobile documentation apparatus
HD height of the display
I inner area of the mask
L length of the housing

1. A mobile documentation apparatus for data detection of skin lesions, for example wounds, which comprises:
a housing;
exactly one, or at least one, recording module, received in the housing or in a part thereof, which comprises:
at least one first camera; and
at least one display for data reproduction or replay and/or for controlling and/or as a user interface for entering control signals for controlling the mobile documentation apparatus.

2. The mobile documentation apparatus according to claim 1, wherein the first camera is a three-dimensional (3D) camera.

3. The mobile documentation apparatus according to claim 1, wherein the mobile documentation apparatus comprises a second camera which is a two-dimensional (2D) camera.

4. The mobile documentation apparatus according to claim 1, comprising, or connected to, at least one evaluation device, configured for collecting data and/or for image processing and comparison to create three-dimensional image models or models of wounds based on the data that was received or measured by the mobile documentation apparatus, in particular configured to control the cameras, sensors, light sources and/or control devices of the mobile documentation apparatus.

5. (canceled)

6. The mobile documentation apparatus according to claim 4, wherein the evaluation device or the recording module or its control are programmed to determine a depth of the wound or lesion to be documented based on the data from the first camera and to forward the result of this determination, e.g., to display it on the display.

7. The mobile documentation apparatus according to claim 4, wherein the evaluation device is physically separate from other components of the mobile documentation apparatus, is in wireless signal communication with at least one of them and/or remotely controls them, or is prepared therefor.

8. (canceled)

9. The mobile documentation apparatus according to claim 4, wherein the evaluation device or another device, e.g., the control device, is programmed to initiate both a 3D image by the first camera and a 2D image by the second camera.

10. (canceled)

11. (canceled)

12. The mobile documentation apparatus according to claim 4, wherein the evaluation device or another device, e.g. the control device, is programmed to
determine a mask from the 2D image, which is, or will be, assigned to the wound in the 2D image, wherein the mask comprises an inner area.

13. The mobile documentation apparatus according to claim 12, wherein the evaluation device is programmed to
specifying a region of the 3D image, which is assigned, after or by the projection of the mask, onto the 3D image to the inner area of the mask, or to an edge or circumferentially closed edge of an area.

14. The mobile documentation apparatus according to claim 13, wherein the evaluation device or another device, e.g., the control device, is programmed to
extract the specified area, or the edge thereof, of the 3D image from the 3D image.

15. The mobile documentation apparatus according to claim 13, wherein the evaluation device or another device, e.g. the control device, is programmed to
evaluate the extracted area.

16. (canceled)

17. (canceled)

18. The mobile documentation apparatus according to claim 15, wherein the evaluation is, or encompasses:
determining the surface area of the wound base lying beneath the surface using the determined distances.

19. The mobile documentation apparatus according to claim 15, wherein the evaluation is, or encompasses:
measuring the extracted area.

20. The mobile documentation apparatus according to claim 1, wherein the mobile documentation apparatus comprises at least one light source for illuminating the skin lesion.

21. The mobile documentation apparatus according to claim 1, wherein the mobile documentation apparatus or its recording module comprises at least one thermal imaging camera.

22. The mobile documentation apparatus according to claim 1, wherein the recording module further comprises at least one sensor, in particular a temperature sensor, a UV camera, an IR sensor and/or a color sensor.

23. The mobile documentation apparatus according to claim 1, further comprising at least one distance sensor.

24. The mobile documentation apparatus according to claim 1, wherein the housing comprises a left holding section and/or a right holding section for the user to hold the mobile documentation apparatus.

25. (canceled)

26. (canceled)

27. (canceled)

28. (canceled)

29. (canceled)

30. (canceled)

31. (canceled)

32. (canceled)

33. The mobile documentation apparatus according to claim 1, wherein the intensity and/or the orientation, or the dimensions of the light cone of the light source, are manually or automatically adjustable.

34. A system encompassing
a mobile documentation apparatus according to claim 1;
a data storage for storing, in particular, images or image data of wounds of one or more patients;
a calculation unit for processing images or image data recorded by the mobile documentation apparatus;
an output device, wherein a device of the system, e.g. the control device, is programmed to output information concerning the recorded images.

35. (canceled)

36. (canceled)

37. (canceled)

38. (canceled)
